(19) United States
(12) Patent Application Publication
(10) Pub. No.: US 2002/0115589 A1
(43) Pub. Date: Aug. 22, 2002

(54) Use of inhibitors of TGF-beta's functions to ameliorate ocular pathology

(76) Inventors: Jon C. Nixon, Mansfield, TX (US); Henry T. Steely JR., Grapevine, TX (US); Herman M. Kunkle, Portsmouth, VA (US); Loretta McNatt, Hurst, TX (US)

Correspondence Address: ALCON RESEARCH, LTD., R&D COUNSEL, Q-148, 6201 SOUTH FREEWAY, FORT WORTH, TX, 76134-2099, US

(21) Appl. No.: 09/992,201

(22) Filed: Nov. 14, 2001

Related U.S. Application Data
(63) Continuation of application No. 09/319,524, filed on Aug. 27, 1999, now abandoned.
(60) Provisional application No. 60/032,667, filed on Dec. 5, 1996.

(30) Foreign Application Priority Data
Dec. 4, 1997 (US) PCT/US97/22282

(51) Int. Cl. A61K 38/17

(52) U.S. Cl. 514/2

(57) ABSTRACT

Compositions comprising at least one TGF-β modulator for treating TGF-β mediated ocular pathologies are disclosed. Methods directed to the treatment of these pathologies, and in particular, glaucoma, ocular hypertension, PVR, secondary cataract, corneal haze and glaucoma filtration surgery bleb failure are also disclosed.

### BACKGROUND OF THE INVENTION

The present invention relates to the field of ophthalmology. In particular, the present invention involves the use of inhibitors or sequesterants of transforming growth factor-beta (“TGF-β”), including the three known isoforms of this molecule occurring in man, to ameliorate various ocular pathologies. More specifically, the compositions and methods are useful in treating glaucoma, proliferative vitreal retinopathy, secondary cataract, corneal haze from post-PRK or anterior chamber surgery, and to suppress scar formation resulting from glaucoma filtration surgery.

The underlying causes of glaucoma are not fully understood. However, it is known that a principal symptom of this disease is elevated intraocular pressure. Elevations of intraocular pressure can ultimately lead to impairment or loss of normal visual function as a result of physical trauma to nerve tissue or ischemia of the supporting vasculature of the retina or optic nerve. It is also known that the elevated intraocular pressure is caused by an excess of fluid (i.e., aqueous humor) within the eye. The excess intraocular fluid is believed to result from blockage or impairment of the normal drainage of fluid from the eye via the trabecular meshwork.

Current drug therapies for treating glaucoma attempt to control intraocular pressure by means of increasing the drainage or “outflow” of aqueous humor from the eye or decreasing the production or “inflow” of aqueous humor by the ciliary processes of the eye. Unfortunately, the use of drug therapy alone is not sufficient to adequately control intraocular pressure in some patients, particularly if there is a severe blockage of the normal outflow passages restricting the movement of aqueous humor out of the eye. Such patients may require surgical intervention to restore the normal outflow of aqueous humor and thereby normalize or at least control their intraocular pressure. The outflow of aqueous humor can be improved by means of glaucoma filtration surgery, wherein a small “bleb” is created on the scleral surface after a fill thickness surgical wound has been made into the anterior chamber to allow the release of excess aqueous humor.

The extracellular matrix (“ECM”) comprises the network of adhesive molecules existing in the extracellular space between cells, including the cells of the trabecular meshwork (“TM”) and cells at or near the glaucoma filtration surgical wound. The ECM regulates the porosity of the TM and attachment of TM cells to the trabecular beams. The ECM also plays an important role in wound structure in glaucoma filtration surgery. Alterations of ECM following filtration surgery may lead to scar formation and ultimate bleb failure. Aberrant expression of ECM component proteins such as fibronectin, collagens, and glycosaminoglycans, has also been noted in the TM of glaucomatous patients, presumably leading to ocular hypertension (Shields, M. B., Primary Open-Angle Glaucoma in Textbook of Glaucoma, 2nd Edition, Williams and Wilkins, pages 151-155 (1987)).

The presence of both TGF-β2, one of the sub-types of TGF-β, in the TM, and thrombospondin (“TSP”) in cultured human TM cells and in the developing mouse eye have been documented (Tripathi et al., Synthesis of a thrombospondin-like cytoadhesion molecule by cells of the trabecular meshwork, Inves. Ophthalmol. Vis Sci., volume 32, pages 181-188 (1991); Rich, K. A., Expression of thrombospondin in the developing mouse eye and cell adhesion of isolated retinal and lens cells, Inves. Ophthalmol. Vis Sci., volume 33 (Supl.), 694 (1992)). Thrombospondin may promote attachment of TM cells to the beams. Latent TGF-β is activated by TSP. Therefore, in the presence of TSP, TGF-β may be converted from a latent to an active form. Specific protease inhibitors for the conversion of the latent form of TGF-β to its active form would also prevent TGF-β action.

It is known that in many tissues, TGF-β(s) stimulates or upregulates the production of major ECM proteins such as fibronectin (“FN”) and its isoforms, collagen, laminin (“LM”), tenancin and/or their respective mRNAs in fibroblasts, epithelial and epithelial-like cells and tissue (Yamamoto et al., Expression of transforming growth factor beta is elevated in human and experimental diabetic nephropathy, Proc. Natl. Acad. Sci., volume 90, pages 1814-1818 (1993); Nakamura et al., Production of extracellular matrix by glomerular epithelial cells is regulated by transforming growth factor beta 1, Kidney Int., volume 41, pages 1213-1221 (1992) and Border et al., Transforming growth factor beta 1 induces extracellular matrix formation in glomerulonephritis, Cell Differ. Dev., volume 32, pages 425-431 (1990)).

TGF-β also differentially regulates the production of ECM proteoglycans such as decorin and biglycan in epithelial cells associated with filtering organs of the body (accessory cells) such as the kidney and liver. (See, Nakamura et al., Production of extracellular matrix by glomerular epithelial cells is regulated by transforming growth factor beta 1, Kidney Int., volume 41, pages 1213-1221 (1992); Vogel et al., The effects of transforming growth factor beta and serum on proteoglycan synthesis by tendon fibrocartilage, Euro. J. Cell Biol., volume 59, pages 304-313 (1992); Meyer et al., Biglycan and decorin gene expression in normal and fibrotic rat liver: cellular location and regulatory factors, Hepatology, volume 16, pages 204-216 (1992); Westergren-Thorsen et al., Transforming growth factor beta induces selective changes in the copolymeric structure of dermatan sulfate in human skin fibroblasts, Eur. J. Biochem., volume 205, pages 277-286 (1992); Westergren-Thorsen et al., The synthesis of a family of structurally related proteoglycans in fibroblasts is differently regulated by TGF-beta, Matrix, volume 11, pages 177-183 (1991) and Romaris et al., Differential effect of transforming growth factor beta on proteglycan synthesis in human embryonic lung fibroblasts, Biochim. Biophys. Acta, volume 1093, pages 229-233 (1991).)

Moreover, TGF-β can regulate both the quantity and type of proteoglycan expressed. For example, TGF-β increases the proportion of D-glucuronosyl residues in human embryonic fibroblasts (Westergren-Thorsen et al., Transforming growth factor beta induces selective changes in the copolymeric structure of dermatan sulfate in human skin fibroblasts, Eur. J. Biochem., volume 205, pages 277-286 (1992)). Proteoglycans are now thought to be the basis of corneal haze formed after trauma to the surface of the eye including laser surgery and dry eye (Rawe et al., A morphological study of rabbit corneas after laser keratectomy, Eye, volume 6 (pt 6), pages 637-642 (1992) and Hanna et al., Corneal stromal wound healing in rabbits after 193-nm excimer laser surface ablation, Arch Ophthalmol., volume 107 (6), pages 895-901 (1989)).

Proliferative vitreoretinopathy (PVR) is a disease characterized by an abnormal growth of fibroblasts into the vitreal chamber. These cells form sheets of fibrous tissue attached to the retina which eventually contract, pulling the retina away from the back of the eye. Fibroblast proliferation and fibrous tissue formation is thought to be mediated in part by elevated levels of TGF-β. This growth factor is a component in so called contraction-stimulating activity of the vitreous collected from patients with PVR at surgery (Hardwick, C., et al., Arch Ophthalmol., volume 113, pages 1545-53 (1995)).

TGF-β levels in the eye are also known to increase during the course of PVR, a disease prevalent in diabetics. TGF-β is thought to play an important role in the progression of this disease by stimulating ECM synthesis, eventually giving rise to pathogenesis associated with hyperproliferation of intravitreal membranes. By sequestering the TGF-β both endogenously synthesized and that secreted by invading macrophages and neutrophils, one might prevent the retinal damage induced by aberrant fibroplasia and ECM which provides a platform for neovascularization.

In summary, the action of TGF-β has been implicated in several ocular pathologies including glaucoma/ocular hypertension, glaucoma filtration surgery bleb failure, secondary cataract, corneal haze and PVR. Therefore, what is needed is a pharmaceutical therapy that would modulate TGF-β in the eye, thereby ameliorating ocular pathologies associated with TGF-β.

Many growth factors (of which TGF-β is one) can be nonspecifically (electrostactically) bound and/or specifically bound to certain specific proteoglycans. In fact, the activity or functional role of the growth factor in the normal cell is most probably modulated by binding of the growth factor to proteoglycans (Ruoslathi et al., Proteoglycans as modulators of growth factor activities, Cell, pages 867-869 (1991)).

In binding growth factors, proteoglycans may serve several roles vital to the functional activity of the growth factor, for example: (a) they protect the factor from proteolytic degradation; (b) they serve as a large reservoir for the growth factor for its immediate delivery to the cell; (c) they prevent the free circulation of unwanted growth factors with the cell's external environment by acting as a molecular “sink” or trap; and (d) they may serve to present the factors in a stereo- or biochemically-specific form to the cell. For example, it has been demonstrated that TGF-β binds very tightly to TSP and in so doing, the growth factor is presented in a biologically active form. This active form suppresses the growth of bovine aortic endothelial cells, a suppression which is not inhibited by the addition of anti-TSP antibodies (Murphy-Ulrich et al., Transforming growth factor beta complexes with thrombospondin, Mol. Cell Biol., volume 3, pages 181-188 (1992). Additionally, Knepper has demonstrated both quantitative and qualitative changes in sulfated glycosaminoglycans, a subset of molecules in the ECM, present in glaucomatous tissue which could theoretically affect binding of TGF-β (Knepper, et al., GAG profile of human TM in primary open angle glaucoma, Inves. Ophthalmol. Vis Sci., volume 30 (Supl.), 224 (1989)).

In vivo experimental models of kidney glomerulonephritis have demonstrated an accumulation of ECM which has been associated with overexpression of TGF-β. Systemic delivery of decorin or biglycan to the kidney and the resultant lowering of systemic blood levels of TGF-β1, inhibits ECM production and dramatically reverses glomerular nephropathy (Border et al., Transforming growth factor beta 1 induces extracellular matrix formation in glomerulonephritis, Cell Differ. Dev., volume 32, pages 425-431 (1990)). This same fibrosis, ECM deposition and resultant kidney dysfunction can also be inhibited by the addition of freely circulating anti-TGF-β antibodies to systemic circulation. Border has suggested that decorin and/or antibodies to TGF-β may be clinically useful in treating renal disease associated with an overproduction of TGF-β (Border et al., Transforming growth factor beta 1 induces extracellular matrix formation in glomerulonephritis, Cell Differ. Dev., volume 32, pages 425-431 (1990)).

### SUMMARY OF THE INVENTION

The present invention provides composition and methods for treating various ocular pathologies. In particular, the present invention is directed to the provision of compositions containing TGF-β blockers, inhibitors, sequesterants or neutralizers and methods of their use in treating glaucoma, scarring associated with glaucoma filtration surgery, corneal haze, secondary cataract, and proliferative vitreoretinopathy.

### DETAILED DESCRIPTION OF THE INVENTION

The present invention provides compositions of blockers, inhibitors, sequesterants or neutralizers of TGF-β and their corresponding methods in treating TGF-β mediated ocular pathologies.

There are five known isoforms of TGF-β. These isoforms have been designated as TGF-β₁, TGF-β₂, TGF-β₃, TGF-β₄ and TGF-β₅, the first three being common to man. The physical properties of these growth factors, sources for their attainment and methods of purification are known. See, for example, U.S. Pat. No. 5,108,989 (Amento, et al; Genentech, Inc.) and the references cited therein at lines 21-45 of column 1. The entire contents of the preceding patent relating to the various forms of TGF-β are hereby incorporated by reference in the present specification. As used herein, the term “TGF-β” encompasses one or more polypeptides from the TGF-β family having the ability to attract fibroblasts and monocytes to surgical sites and mitogenically activate these cells.

While not intending to be bound by any theory, it is believed that the inappropriate presence of TGF-β in the ECM of the TM and other tissues of the eye creates a risk factor for glaucoma. It is also believed that inappropriate amounts of TGF-β in the vitreous of the eye affects cellular proliferation leading to PVR. It is further believed that inappropriate amounts of TGF-β also affect corneal haze and secondary cataract following surgery. Therefore, modulation of TGF-β in the ocular tissues to which it is acting as a pathogen may ameliorate any of the above described conditions.

There are numerous ways in which TGF-β can be modulated. TGF-β activity may be inhibited by an antagonist directed to the TGF-β receptors. TGF-β activity may be inhibited by binding TGF-β with normal extracellular components. TGF-β may also be “sequestered,” i.e. tightly bound, and therefore made inactive, by proteins with high affinity for TGF-β. As used herein, the term “TGF-β modulators” refers to one or more compound(s), protein(s), or combination which neutralizes or diminishes the pathological effect of TGF-β in the eye.

TGF-β may be modulated by proteoglycans. Proteoglycans are heavily glycosylated proteins either freely soluble or found in the ECM. Examples of proteoglycans include decorin, biglycan, lumican, and fibromodulin. As used herein, the term “proteoglycan” refers to proteins with at least one glycosaminoglycan side chain.

TGF-β may be modulated by the antibodies or fab-fragments of antibodies directed to TGF-β. By binding specific sites of activity on TGF-β, the antibody serves to prevent binding of TGF-β to its cognate cellular receptor. Thus, bound TGF-β would be rendered inactive and therefore, unable to perform its deleterious effects.

TGF-β may also be modulated by receptors or fragments of receptors to TGF-β. These receptors normally reside on various cellular surfaces and bind TGF-β, thereby facilitating cellular responses. The use of these receptors and fragments in a solubilized form (i.e., not part of a membrane structure) can be employed to bind TGF-β and sequester it from its targeted biological action.

TGF-β may also be modulated by purified serum proteins such as α2-macroglobulins. The proteins may be formulated for use during surgery or for topical therapy to sequester and/or prevent the activation of TGF-β (Schulz et al., Inhibition of transforming growth factor-β-induced cataractous changes in lens explants by ocular media and α2-macroglobulin, Investigative Ophthalmology & Visual Science, volume 37, no. 8, pages 1509-1519 (1996)).

The TGF-β modulators may be contained in various types of pharmaceutical compositions in accordance with formulation techniques known to those skilled in the art. The route of administration (e.g., topical or intraocular) and the dosage regimen will be determined by skilled clinicians, based on factors such as the exact nature of the condition being treated, the severity of the condition, the age and general physical condition of the patient, and so on.

The method of administration of TGF-β modulators will depend on the disease to be treated and other factors such as the duration of therapy and whether the modulators will be administered prophylactally or during acute phases such as surgery. The TGF-β modulators may be used as an adjunct to ophthalmic surgery, such as by vitreal or subconjunctival injection following ophthalmic surgery. The compounds may be used for acute treatment of temporary conditions, or may be administered chronically, especially in the case of degenerative disease. The compounds may also be used prophylactically, especially prior to ocular surgery or non-invasive ophthalmic procedures, or other types of surgery.

When treating glaucoma by means other than surgery, TGF-β modulators generally will be formulated and administered for topical application. Topical formulations are generally aqueous in nature, buffered to a physiological acceptable pH and typically preserved for multi-dispensing.

The topical ophthalmic compositions of the present invention will include one or more TGF-β modulators and a pharmaceutically acceptable vehicle for said compound(s). Various types of vehicles may be utilized. The vehicles will generally be aqueous in nature. Aqueous solutions are generally preferred, based on ease of formulation, as well as patients' ability to easily administer such compositions by means of instilling one to two drops of the solutions in the affected eyes. However, the TGF-β modulators may also be readily incorporated into other types of compositions, such as suspensions, viscous or semi-viscous gels or other types of solid or semi-solid compositions. Suspensions may be preferred for TGF-β modulators which are relatively insoluble in water. The ophthalmic compositions of the present invention may also include various other ingredients, such as buffers, preservatives, co-solvents and viscosity building agents.

An appropriate buffer system (e.g., sodium bicarbonate, sodium phosphate, sodium acetate, sodium citrate, sodium ascorbate or sodium borate) may be added to prevent pH drift under storage conditions.

Ophthalmic products are typically packaged in multidose form. Preservatives are thus required to prevent microbial contamination during use. Suitable preservatives include, for example: benzalkonium chloride, thimerosal, chlorobutanol, methyl paraben, propyl paraben, phenylethyl alcohol, edetate disodium, sorbic acid, polyquaternium-1, or other agents known to those skilled in the art. Such preservatives are typically employed at a level of from 0.001 to 1.0 percent by weight, based on the total weight of the composition (wt. %).

Some of the compounds of the TGF-β modulators may have limited solubility in water and therefore may require a surfactant or other appropriate co-solvent in the composition. Such co-solvents include, for example: polyethoxylated castor oils, Polysorbate 20, 60 and 80; Pluronic® F-68, F-84 and P-103 (BASF Corp., Parsippany N.J., USA); cyclodextrins; or other agents known to those skilled in the art. Such co-solvents are typically employed at a level of from 0.01 to 2 wt. %.

Viscosity greater than that of simple aqueous solutions may be desirable to increase ocular absorption of the active compound, to decrease variability in dispensing the formulations, to decrease physical separation of components of a suspension or emulsion of formulation and/or otherwise to improve the ophthalmic formulation. Such viscosity building agents include, for example, polyvinyl alcohol, polyvinyl pyrrolidone, methyl cellulose, hydroxypropyl methylcellulose, hydroxyethyl cellulose, carboxymethyl cellulose, hydroxypropyl cellulose or other agents known to those skilled in the art. Such agents are typically employed at a level of from 0.01 to 2 wt. %.

When treating PVR, the TGF-β modulators will be formulated for intraocular use. Such formulations generally will comprise a surgical irrigating solution such as a fluornated hydrocarbon in BSS Plus® Sterile Irrigating Solution or BSS Plus® Sterile Irrigating Solution alone, as described below.

The use of physiologically balanced irrigating solutions as pharmaceutical vehicles for the TGF-β modulators is preferred when the compounds are administered intraocularly. As utilized herein, the term “physiologically balanced irrigating solution” means a solution which is adapted to maintain the physical structure and function of tissues during invasive or noninvasive medical procedures. This type of solution will typically contain electrolytes, such as sodium, potassium, calcium, magnesium and/or chloride; an energy source, such as dextrose; and a buffer to maintain the pH of the solution at or near physiological levels. Various solutions of this type are known (e.g., Lactated Ringers Solution). BSS® Sterile Irrigating Solution and BSS Plus® Sterile Intraocular Irrigating Solution (Alcon Laboratories, Inc., Fort Worth, Tex., USA) are examples of physiologically balanced intraocular irrigating solutions. The latter type of solution is described in U.S. Pat. No. 4,550,022 (Garabedian, et al.), the entire contents of which are hereby incorporated in the present specification by reference.

The doses utilized for any of the above-described purposes of topical, periocular or intraocular administration will generally be from about 0.01 to about 100 milligrams per kilogram of body weight (mg/kg), administered one to four times per day. As used herein, the term “pharmaceutically effective amount” refers to that amount of a TGF-β modulator(s) which modulates TGF-β in the eye to such a level that treatment of the ocular condition is ameliorative. As used herein, the term “pharmaceutically acceptable carrier” refers to any formulation which is safe and provides an effective delivery of an effective amount of at least one TGF-β modulator to the target tissue.

The compositions of the present invention are further illustrated by the following formulation examples:

### EXAMPLE 1

Topical Compositions Useful for Modulating TGF-β:

| Component | wt. % |
|---|---|
| TGF-β Modulator | 0.005-5.0 |
| Tyloxapol | 0.01-0.05 |
| HPMC | 0.5 |
| Benzalkonium Chloride | 0.01 |
| Sodium Chloride | 0.8 |
| Edetate Disodium | 0.01 |
| NaOH/HCl | q.s. pH 7.4 |
| Purified Water | q.s. 100 mL |

### EXAMPLE 2

Formulation for Sterile Intraocular Injection:

| Component | each mL contains: | |
|---|---|---|
| TGF-β Modulator | 10-100 | mg |
| Sodium Chloride | 7.14 | mg |
| Potassium Chloride | 0.38 | mg |
| Calcium chloride dihydrate | 0.154 | mg |
| Magnesium chloride hexahydrate | 0.2 | mg |
| Dried sodium phosphate | 0.42 | mg |
| Sodium bicarbonate | 2.1 | mg |
| Dextrose | 0.92 | mg |

| | |
|---|---|
| Hydrochloric acid or sodium | q.s., pH to approx. 7.2 |
| hydroxide | |
| Water for injection | q.s. |

### EXAMPLE 3

Preferred Formulation for a Topical Ocular Solution:

| Component | wt. % |
|---|---|
| TGF-β Modulator | 1.0 % |
| Benzalkonium chloride | 0.01 % |
| HPMC | 0.5 % |
| Sodium chloride | 0.8 % |
| Sodium phosphate | 0.28 % |
| Edetate disodium | 0.01 % |
| NaOH/HCl | q.s. pH 7.2 |
| Purified Water | q.s. 100 mL |

What is claimed is:

1. A composition for treating TGF-β mediated ocular pathologies in the eye comprising a pharmaceutical effective amount of at least one TGF-β modulator in a pharmaceutically acceptable vehicle.

2. A composition according to claim 1, wherein the composition is a topical or intraocular formulation.

3. A composition according to claim 1, wherein the TGF-β modulator(s) are selected from the group consisting of: decorin, biglycan, fibromodulin, lumican, epiphycan, versican, aggrecan, neurocan, brevican, perlecan, agrin, testican and α-macroglobulin.

4. A composition according to claim 3, wherein the TGF-β modulator(s) are selected from group consisting of decorin, lumican and α-macroglobulin.

5. A composition according to claim 4, wherein the TGF-β modulator is decorin.

6. A method for treating TGF-β mediated ocular pathologies in the eye which comprises administering a composition comprising a pharmaceutically effective amount of at least one TGF-β modulator to the eye.

7. A method according to claim 6, wherein the ocular pathologies to be treated are selected from the group consisting of: glaucoma, ocular hypertension, PVR, secondary cataract, corneal haze and glaucoma filtration surgery bleb failure.

8. A method according to claim 6, wherein the composition is a topical or intraocular formulation.

9. A method according to claim 6, wherein the TGF-β modulator(s) is selected from the group consisting of: decorin, biglycan, fibromodulin, lumican, epiphycan, versican, aggrecan, neurocan, brevican, perlecan, agrin, testican and α-macroglobulin.

10. A method according to claim 9, wherein the TGFβ modulator(s) are selected from group consisting of decorin, lumican and α-macroglobulin.

11. A method according to claim 10, wherein the TGF-β modulator is decorin.
